(19) United States
(12) Patent Application Publication
(10) Pub. No.: US 2005/0187439 A1
(43) Pub. Date: Aug. 25, 2005

(54) Sampling interface system for in-vivo estimation of tissue analyte concentration

(76) Inventors: Thomas B. Blank, Gilbert, AZ (US); George M. Acosta, Murrieta, CA (US); Stephen L. Monfre, Gilbert, AZ (US); Roxanne E. Abul-Haj, Mesa, AZ (US); Kevin H. Hazen, Gilbert, AZ (US); Sedar Brown, Phoenix, AZ (US); Benjamin L. Richie JR., Scottsdale, AZ (US); James R. Henderson, Phoenix, AZ (US); Barry C. Elliott, Phoenix, AZ (US); Josh Hope, Gilbert, AZ (US)

Correspondence Address: GLENN PATENT GROUP, 3475 EDISON WAY, SUITE L, MENLO PARK, CA, 94025, US

(21) Appl. No.: 11/031,103

(22) Filed: Jan. 6, 2005

Related U.S. Application Data
(60) Provisional application No. 60/536,197, filed on Jan. 12, 2004.
(60) Provisional application No. 60/534,834, filed on Jan. 6, 2004.
(60) Provisional application No. 60/566,568, filed on Apr. 28, 2004.

(30) Foreign Application Priority Data
Mar. 7, 2003 (WO) PCT/US03/07065

(51) Int. Cl. A61B 5/00

(52) U.S. Cl. 600/310; 600/344

(57) ABSTRACT

Sampling is controlled to enhance analyte concentration estimation derived from noninvasive sampling. Means of assuring that the same tissue sample volume is repeatably sampled are presented, thus minimizing sampling errors due to mechanical tissue distortion, specular reflectance, and probe placement. In a first embodiment of the invention, sampling is controlled using automated delivery of a coupling fluid to a region between a tip of a sample probe and a tissue measurement site in a manner requiring minimal user interaction. In a second embodiment of the invention, sampling is controlled by controlling temperature variations, preferably with a coupling fluid, at a region about the tip of a sample probe and a sample site. In a third embodiment, sampling is procedurally controlled via timing and location of coupling fluid delivery to a sample site.

### CROSS REFERENCES TO RELATED APPLICATIONS

This application claims priority from:
U.S. provisional patent application Ser. No. 60/536,197, filed Jan. 12, 2004;
U.S. provisional patent application Ser. No. 60/534,834, filed Jan. 6, 2004;
U.S. provisional patent application Ser. No. 60/566,568, filed Apr. 28, 2004;
U.S. patent application Ser. No. 10/472,856, filed Mar. 7, 2003, which claims priority from PCT application no. PCT/US03/07065, filed Mar. 7, 2003, which claims benefit of U.S. provisional patent application Ser. No. 60/362,885, filed Mar. 8, 2002; and
U.S. patent application Ser. No. 10/170,921 filed Jun. 12, 2002, which claims benefit of U.S. patent application Ser. No. 09/563,782, now U.S. Pat. No. 6,415,167, which issued Jul. 2, 2002 each of which is incorporated herein in its entirety by this reference thereto.

### BACKGROUND OF THE INVENTION

1. Field of the Invention

This invention relates generally to the noninvasive measurement of biological parameters through near-infrared spectroscopy. More particularly, a method and apparatus are disclosed for the automated delivery of a coupling fluid between an analyzer and a tissue sample site, for thermal control of a sample site, or for integrated delivery of a coupling fluid in a sampling process.

2. Discussion of the Prior Art

### TECHNICAL BACKGROUND

In-vivo measurement of tissue properties or analyte concentration using optical based analyzers requires that a tissue measurement region be positioned and coupled with respect to an optical interface or probe, such as a tip of a sampling module. The requirements of a sampling interface system for probe placement and coupling depends upon the nature of the tissue properties and analytes under consideration, the optical technology being applied, and the variability of the tissue sample site. There are many demanding in-vivo applications that require a high degree of sampling reproducibility. In one example, a relatively unskilled operator or user must perform the optical measurement. One exemplary application is the noninvasive estimation of glucose concentration through near-infrared spectroscopy in a variety of environments. This problem is further considered through a discussion of the target application and the structure, variability, and dynamic properties of live tissue.

Diabetes

Diabetes is a chronic disease that results in abnormal production and use of insulin, a hormone that facilitates glucose uptake into cells. While a precise cause of diabetes is unknown, genetic factors, environmental factors, and obesity play roles. Diabetics have increased risk in three broad categories: cardiovascular heart disease, retinopathy, and neuropathy. Diabetics often have one or more of the following complications: heart disease and stroke, high blood pressure, kidney disease, neuropathy (nerve disease and amputations), retinopathy, diabetic ketoacidosis, skin conditions, gum disease, impotence, and fetal complications. Diabetes is a leading cause of death and disability worldwide. Moreover, diabetes is merely one among a group of disorders of glucose metabolism that also includes impaired glucose tolerance and hyperinsulinemia, which is also known as hypoglycemia.

Diabetes Prevalence and Trends

The prevalence of individuals with diabetes is increasing with time. The World Health Organization (WHO) estimates that diabetes currently afflicts 154 million people worldwide. There are 54 million people with diabetes living in developed countries. The WHO estimates that the number of people with diabetes will grow to 300 million by the year 2025. In the United States, 15.7 million people or 5.9 percent of the population are estimated to have diabetes. Within the United States, the prevalence of adults diagnosed with diabetes increased by 6% in 1999 and rose by 33% between 1990 and 1998. This corresponds to approximately eight hundred thousand new cases every year in America. The estimated total cost to the United States economy alone exceeds $90 billion per year. Diabetes Statistics, National Institutes of Health, Publication No. 98-3926, Bethesda, Md. (November 1997).

Diabetes Treatment

Long-term clinical studies show that the onset of diabetes related complications are significantly reduced through proper control of blood glucose concentrations [The Diabetes Control and Complications Trial Research Group, The effect of intensive treatment of diabetes on the development and progression of long-term complications in insulin-dependent diabetes mellitus, N Eng J of Med 1993;329:977-86; U.K. Prospective Diabetes Study (UKPDS) Group, Intensive blood-glucose control with sulphonylureas or insulin compared with conventional treatment and risk of complications in patients with type 2 diabetes, Lancet, vol. 352, pp. 837-853 (1998); Ohkubo, Y., H. Kishikawa, E. Araki, T. Miyata, S. Isami, S. Motoyoshi, Y. Kojima, N. Furuyoshi, and M. Shichizi, Intensive insulin therapy prevents the progression of diabetic microvascular complications in Japanese patients with non-insulin-dependent diabetes mellitus: a randomized prospective 6-year study, Diabetes Res Clin Pract, vol. 28, pp. 103-117, (1995)]. A vital element of diabetes management is the self-monitoring of blood glucose concentrations or levels by diabetics in the home environment. However, current monitoring techniques discourage regular use due to the inconvenient and painful nature of drawing blood through the skin prior to analysis (The Diabetes Control and Complication Trial Research Group, The effect of intensive treatment of diabetes on the development and progression of long-term complications of insulin-dependent diabetes mellitus, supra. Unfortunately, recent reports indicate that even periodic measurement of glucose concentration by individuals with diabetes, (e.g. seven times per day) is insufficient to detect important glucose concentration fluctuations and properly manage the disease. Therefore, a device that provides noninvasive, automatic, and/or nearly continuous estimations of glucose concentrations is of substantial value to people with diabetes. Implantable glucose concentration analyzers eventually coupled to an insulin delivery system providing an artificial pancreas are also being pursued.

Noninvasive Glucose Concentration Estimation

There exist a number of noninvasive approaches for glucose concentration estimation. These approaches vary widely, but have at least two common steps. First, an apparatus is used to acquire a reading from the body without obtaining a biological sample for every glucose concentration estimation. Second, an algorithm is used to convert the noninvasive reading into a glucose concentration estimation.

Several terms or phrases are loosely used in the literature as synonymous with the phrase glucose concentration estimation. These alternative phrases include: measurement of glucose, measurement of glucose concentration, glucose determination, and glucose concentration determination. The term measurement or determination is most appropriately used with a technique that is more directly tied to glucose, such as gravimetric or electroenzymatic techniques. Glucose concentration estimation is the preferable term for use with indirect techniques or techniques based upon soft models and is the term used herein.

Technologies

A number of previously reported technologies for estimating glucose concentration noninvasively exist that involve the measurement of a tissue related variable. One species of noninvasive glucose concentration analyzer uses spectroscopy to acquire a signal or spectrum from the body. Examples include far-infrared absorbance spectroscopy, tissue impedance, Raman, and fluorescence, as well as techniques using light from the ultraviolet through the infrared [ultraviolet (200 to 400 nm), visible (400 to 700 nm), near-infrared (700 to 2500 nm or 14,286 to 4000 cm⁻¹), and mid-infrared (2500 to 14,285 nm or 4000 to 700 cm⁻¹)]. Notably, noninvasive techniques do not have to be based upon spectroscopy. For example, a bioimpedence meter is a noninvasive device. In this document, any device that reads glucose concentration from the body without penetrating the skin and collecting a biological sample with each sample is referred to as a noninvasive glucose concentration analyzer. For the purposes of this document, X-rays and magnetic resonance imagers (MRI's) are not considered to be defined in the realm of noninvasive technologies.

It is important to note that noninvasive techniques are distinct from invasive techniques in that the sample analyzed is a portion of the human body in-situ, not a biological sample acquired from the human body. The actual tissue volume that is sampled is the portion of irradiated tissue from which light is diffusely reflected, transflected, or diffusely transmitted to the spectrometer detection system. Noninvasive techniques share the common characteristic that a calibration is required to derive a glucose concentration from subsequent data collection. An example of a particular range for noninvasive glucose concentration estimation in diffuse reflectance mode is about 1100 to 2500 nm or ranges therein, such as 1200 to 1800 nm. K. Hazen, Glucose determination in biological matrices using near-infrared spectroscopy, doctoral dissertation, University of Iowa, 1995 describes additional near-infrared ranges typically used for noninvasive glucose concentration estimation.

Instrumentation

A number of spectrometer configurations are reported for collecting noninvasive spectra of regions of the body. Typically a spectrometer has one or more beam paths from a source to a detector. Optional light sources. include a blackbody source, a tungsten-halogen source, one or more light emitting diodes, or one or more laser diodes. For multi-wavelength spectrometers a wavelength selection device is optionally used or a series of optical filters are optionally used for wavelength selection. Wavelength selection devices include dispersive elements, such as one or more plane, concave, ruled, or holographic grating. Additional wavelength selective devices include an interferometer, successive illumination of the elements of a light emitting diode array, prisms, and wavelength selective filters. Optionally, variation of the source output, such as varying which light emitting diode or laser diode is firing, is used. Detectors are in the form of one or more single element detectors or one or more arrays or bundles of detectors. Optional detectors include at least indium gallium arsenide (InGaAs), extended indium gallium arsenide, lead sulfide (PbS), lead selenide (PbSe), silicon (Si), mercury cadmium telluride (MCT), or the like. Detectors optionally further include arrays of InGaAs, extended InGaAs, PbS, PbSe, Si, MCT, or the like. Light collection optics, such as fiber optics, lenses, and mirrors, are commonly used in various configurations within a spectrometer to direct light from the source to the detector by way of a sample. The mode of operation is diffuse transmission, diffuse reflectance, and/or transflectance.

Due to changes in performance of the overall spectrometer, reference wavelength standards and/or intensity standards are often used. Typically, a wavelength standard is collected immediately before or after the interrogation of the tissue or at the beginning of a sampling period, such as a few hours or a day. Optionally wavelength standard spectra are obtained at times far removed from the period of sampling, such as when the spectrometer was originally manufactured, or on a weekly or monthly basis. A typical reference wavelength standard is polystyrene or a rare earth oxide, such as holmium, erbium, or dysprosium oxide. Many additional materials exist that have stable and sharp spectral features that are optionally used as a reference standard.

Sampling

Light is directed to and from a glucose concentration analyzer to a tissue sample site by optical methods, such as through a light pipe, fiber-optics, a lens system, free space optics, and/or a light directing mirror system. Typically, one or more of three modes are used to collect noninvasive scans: transmittance, transflectance, and/or diffuse reflectance. Collected signal is converted to a voltage and sampled through an analog-to-digital converter for analysis on a microprocessor based system and the result displayed.

The sample site is the specific tissue of the subject that is irradiated with incident light that is subsequently detected. The sample site surface of the subject is the region that the measurement probe comes into contact with. Ideal qualities of the sample site include homogeneity, immutability, and accessibility to the target analyte. Noninvasive sample sites include regions or volumes of the body, such as a hand, finger, palmar region, base of thumb, forearm, volar aspect of the forearm, dorsal aspect of the forearm, upper arm, head, earlobe, eye, tongue, chest, torso, abdominal region, thigh, calf, foot, plantar region, and toe.

Human Tissue/Light Interaction

When incident light is directed onto the skin surface, a part of it is reflected while the remaining part penetrates the skin surface. The proportion of reflected light energy is strongly dependent on the angle of incidence. At nearly perpendicular incidence, about four percent of the incident beam is reflected due to the change in refractive index between air (η_{D}=1.0) and dry stratum corneum (η_{D}=1.55). For normally incident radiation, this specular reflectance component is as high as seven percent, because the very rigid and irregular surface of the stratum corneum produces off-normal angles of incidence. Regardless of skin color, specular reflectance of a nearly perpendicular beam from normal skin ranges between four and seven percent over the entire spectrum from 250 to 3000 nm. See R. Scheuplein, J. Soc. Cosmet. Chem., v.15, pp. 111-122 (1964). The air-stratum corneum border gives rise to a regular reflection. Results indicate that the indices of refraction of most soft tissue (skin, liver, heart, etc) lie within the 1.38-1.41 range with the exception of adipose tissue, which has a refractive index of approximately 1.46. See J. Parrish, R. Anderson, F. Urbach, D. Pifts, UV-A: Biologic Effects of Ultraviolet Radiation with Emphasis on Human Responses to Longwave Ultraviolet, New York, Plenum Press (1978). Therefore, these differences in refractive index between the different layers of the skin are generally too small to give a noticeable reflection. See Ebling, supra. The differences are expected to be even more insignificant when the stratum corneum is hydrated, owing to refractive index matching. The 93 to 96 percent of the incident beam that enters the skin is attenuated due to absorption and/or scattering within any of the layers of the skin. These two processes taken together essentially determine the penetration of light into skin, as well as remittance of scattered light from the skin.

There are a number of reports of noninvasive glucose technologies. Some of these relate to general instrumentation configurations required for noninvasive glucose concentration estimation while others refer to sampling technologies. Those related to the present invention are briefly reviewed, infra.

General Instrumentation

Pulse oximeters operate on wavelengths about 660 and 805 nm, which correlate to oxy-hemoglobin and deoxy-hemoglobin absorbance bands. Siemens, AG, Verfahren und Gerät zur kolorimetrischen Untersuchung von Substanzen auf signifikante Bestandteile (Method and device for a colorimetric examination of substances for significant components), DE 2,255,300, filed Nov. 11, 1972 describes a pulse oximeter meter operating in a spectral region of 600 to 900 nm, which is at shorter wavelengths than the noninvasive glucose concentration meters of this invention that operate from about 1100 to 2500 nm or ranges therein.

K. Schlager, Non-invasive near infrared measurement of blood analyte concentrations, U.S. Pat. No. 4,882,492, (Nov. 21, 1989) describes a dual beam noninvasive glucose analyzer.

P. Rolfe, Investigating substances in a patient's bloodstream, U.K. patent application ser. no. 2,033,575 (Aug. 24, 1979) describes an apparatus for directing light into the body, detecting attenuated backscattered light, and using the collected signal to estimate glucose concentrations in or near the bloodstream.

C. Dahne, D. Gross, Spectrophotometric method and apparatus for the non-invasive, U.S. Pat. No. 4,655,225 (Apr. 7, 1987) describe a method and apparatus for directing light into a patient's body, collecting transmitted or backscattered light, and estimating glucose concentrations from selected near-infrared (near-IR) wavelength bands. Wavelengths regions include 1560 to 1590, 1750 to 1780, 2085 to 2115, and 2255 to 2285 nm, with at least one additional reference signal from 1000 to 2700 nm.

J. Hall, T. Cadell, Method and device for measuring concentration levels of blood constituents non-invasively, U.S. Pat. No. 5,361,758 (Nov. 8, 1994) describe a noninvasive device and method for estimating analyte concentrations within a living subject using polychromatic light, a wavelength separation device, and an array detector. The apparatus uses a receptor shaped to accept a fingertip with means for blocking extraneous light.

J. Garside, S. Monfre, B. Elliott, T. Ruchti, G. Kees, Fiber optic illumination and detection patterns, shapes, and locations for use in spectroscopic analysis, U.S. Pat. No. 6,411,373, (Jun. 25, 2002) describe the use of fiber optics for use as excitation and/or collection optics with various spatial distributions.

Specular Reflectance

R. Messerschmidt, D. Sting, Blocker device for eliminating specular reflectance from a diffuse reflectance spectrum, U.S. Pat. No. 4,661,706 (Apr. 28, 1987) describe a reduction of specular reflectance by a mechanical device. A blade-like device “skims” the specular light before it impinges on the detector. This system leaves alignment concerns and improvement in efficiency of collecting diffusely reflected light is needed.

R. Messerschmidt, M. Robinson, Diffuse reflectance monitoring apparatus, U.S. Pat. No. 5,636,633 (Jun. 10, 1997) describe a specular control device for diffuse reflectance spectroscopy using a group of reflecting and open sections.

R. Messerschmidt, M. Robinson, Diffuse reflectance monitoring apparatus, U.S. Pat. No. 5,935,062 (Aug. 10, 1999) and R. Messerschmidt, M. Robinson, Diffuse reflectance monitoring apparatus, U.S. Pat. No. 6,230,034 (May 8, 2001) describe a diffuse reflectance control device that discriminates between diffusely reflected light that is reflected from selected depths. This control device additionally acts as a blocker to prevent specularly reflected light from reaching the detector.

S. Malin, G Khalil, Method and apparatus for multi-spectral analysis of organic blood analytes in noninvasive infrared spectroscopy, U.S. Pat. No. 6,040,578 (Mar. 21, 2000) describe the use of specularly-reflected light in regions of high water absorbance, such as 1450 and 1900 nm, to mark the presence of outlier spectra wherein the specularly reflected light is not sufficiently reduced.

K. Hazen, G. Acosta, A. Abul-Haj, R. Abul-Haj, Apparatus and method for reproducibly modifying localized absorption and scattering coefficients at a tissue measurement site during optical sampling, U.S. Pat. No. 6,534,012 (Mar. 18, 2003) describe a mechanical device for applying sufficient and reproducible contact of the apparatus to the sample medium to minimize specular reflectance. Further, the apparatus allows for reproducible applied pressure to the sample site and reproducible temperature at the sample site.

Temperature

K. Hazen, Glucose determination in biological matrices using near-Infrared spectroscopy, doctoral dissertation, University of Iowa (1995) describes the adverse effect of temperature on near-infrared based glucose concentration estimations. Physiological constituents have near-infrared absorbance spectra that are sensitive, in terms of magnitude and location, to localized temperature and the sensitivity impacts noninvasive glucose concentration estimation.

Coupling Fluid

A number of sources describe coupling fluids as a consideration in noninvasive sampling methods and apparatus. Coupling fluids have been long known and understood in the field of optics. Some coupling fluids are used to fill optical irregularities. Others are used for refractive index matching. Some, such as glycerol when used in conjunction with near-infrared light, absorb in the wavelength region of interest. Several reports of optical coupling fluids and a report of a coupling fluid are described, infra.

R. Messerschmidt, Method for non-Invasive blood analyte measurement with improved optical interface, U.S. Pat. No. 5,655,530, Aug. 12, 1997 and R. Messerschmidt, Method for non-invasive blood analyte measurement with improved optical interface, U.S. Pat. No. 5,823,951, (Oct. 20, 1998) describe an index-matching medium to improve the interface between a sensor probe and a skin surface during spectrographic analysis. These patents teach an optical coupling medium containing perfluorocarbons and chlorofluorocarbons that have minimal absorbance in the near-infrared. Since they are known carcinogens, chlorofluorocarbons (CFC's) are unsuitable for use in preparations to be used on living tissue. Furthermore, use of CFC's poses a well-known environmental risk. Additionally, Messerschmidt's interface medium is formulated with substances that are likely to leave artifacts in spectroscopic measurements.

M. Robinson, R. Messerschmidt, Method for non-invasive blood analyte measurement with improved optical interface, U.S. Pat. No. 6,152,876 (Nov. 28, 2000) and M. Rohrscheib, C. Gardner, M. Robinson, Method and apparatus for non-invasive blood analyte measurement with fluid compartment equilibration, U.S. Pat. No. 6,240,306 (May 29, 2001) describe an index-matching optical coupling fluid used to improve the interface between the sensor probe and skin surface during spectroscopic analysis. The index-matching medium is preferably a composition containing chlorofluorocarbons with optional perfluorocarbons.

T. Blank, G. Acosta, M. Maftu, S. Monfre, Fiber optic probe guide placement guide, U.S. Pat. No. 6,415,167 (Jul. 2, 2002) describe a coupling fluid of one or more perfluoro compounds where a quantity of the coupling fluid is placed at an interface of the tip of an optical probe of a sample module and a measurement site. Advantageously, perfluoro compounds lack the toxicity associated with chlorofluorocarbons.

Pressure

E. Chan, B. Sorg, D. Protsenko, M. O'Neil, M. Motamedi, A. Welch, Effects of compression on soft tissue optical properties, IEEE Journal of Selected Topics in Quantum Electronics, Vol. 2, no. 4, 943-950 (1996) describe the effect of pressure on absorption and reduced scattering coefficients from 400 to 1800 nm. Most specimens show an increase in the scattering coefficient with compression.

K. Hazen, G. Acosta, A. Abul-Haj, R. Abul-Haj, Apparatus and method for reproducibly modifying localized absorption and scattering coefficients at a tissue measurement site during optical sampling, U.S. Pat. No. 6,534,012 (Mar. 18, 2003) describe in a first embodiment a noninvasive glucose concentration estimation apparatus for either varying the pressure applied to a sample site or maintaining a constant pressure on a sample site in a controlled and reproducible manner by moving a sample probe along the z-axis perpendicular to the sample site surface. In an additional described embodiment, the arm sample site platform is moved along the z-axis that is perpendicular to the plane defined by the sample surface by raising or lowering the sample holder platform relative to the analyzer probe tip. The '012 patent further teaches proper contact between the probe tip and the sample site to be that point at which specularly-reflected light is substantially zero at the water bands at 1950 and 2500 nm. M. Makarewicz, M. Mattu, T. Blank, G. Acosta, E. Handy, W. Hay, T. Stippick, B. Richie, Method and apparatus for minimizing spectral interference due to within and between sample variations during in-situ spectral sampling of tissue, U.S. patent application Ser. No. 09/954,856 (filed Sep. 17, 2001) describe a temperature and pressure controlled sample interface. The means of pressure control is a set of supports for the sample that control the natural position of the sample probe relative to the sample.

Data Processing

Several approaches exist that employ diverse preprocessing methods to remove spectral variation related to the sample and instrument variation including normalization, smoothing, derivatives, multiplicative signal correction, [P. Geladi, D. McDougall, H. Martens Linearization and scatter-correction for near-infrared reflectance spectra of meat, Applied Spectroscopy, vol. 39, 491-500, (1985)], standard normal variate transformation, [R. Barnes, M. Dhanoa, S. Lister, Applied Spectroscopy, 43, 772-777, (1989)], piecewise multiplicative scatter correction, [T. Isaksson and B. Kowalski, Applied Spectroscopy, 47, 702-709, (1993)], extended multiplicative signal correction, [H. Martens, E. Stark, J. Pharm Biomed Anal, 9, 625-635, (1991)], pathlength correction with chemical modeling and optimized scaling, [GlucoWatch automatic glucose biographer and autosensors, Cygnus Inc., Document #1992-00, Rev. March (2001)], and finite impulse response filtering, [S. Sum, Spectral signal correction for multivariate calibration, Doctoral Dissertation, University of Delaware, (1998); S. Sum, S. Brown, Standardization of fiber-optic probes for near-infrared multivariate Calibrations, Applied Spectroscopy, Vol. 52, No. 6, 869-877, (1998); and T. Blank, S. Sum, S. Brown, S. Monfre, Transfer of near-infrared multivariate calibrations without standards, Analytical Chemistry, 68, 2987-2995, (1996)].

In addition, a diversity of signal, data, or pre-processing techniques are commonly reported with the fundamental goal of enhancing accessibility of the net analyte signal [D. Massart, B. Vandeginste, S. Deming, Y. Michotte, L. Kaufman, Chemometrics: a textbook, New York, Elsevier Science Publishing Company, Inc., 215-252, (1990); A. Oppenheim, R. Schafer, Digital Signal Processing, Englewood Cliffs, N.J.: Prentice Hall, 1975, 195-271; M. Otto, Chemometrics, Weinheim: Wiley-VCH, 51-78, (1999); K. Beebe, R. Pell, M. Seasholtz, Chemometrics A Practical Guide, New York: John Wiley & Sons, Inc., 26-55, (1998); M. Sharaf, D. Illman and B. Kowalski, Chemometrics, New York: John Wiley & Sons, Inc., 86-112, (1996); and A. Savitzky, M. Golay, Smoothing and differentiation of data by simplified least squares procedures, Anal. Chem., vol. 36, no. 8, 1627-1639, (1964)]. A goal of these techniques is to attenuate the noise and instrument variation while maximizing the signal of interest.

While methods for preprocessing partially compensate for variation related to instrument and physical changes in the sample and enhance the net analyte signal in the presence of noise and interference, they are often inadequate for compensating for the sources of tissue-related variation. For example, the highly nonlinear effects related to sampling different tissue locations are not effectively compensated for through a pathlength correction because the sample is multi-layered and heterogeneous. In addition, fundamental assumptions inherent in these methods, such as the constancy of multiplicative and additive effects across the spectral range and homoscadasticity of noise are violated in the noninvasive tissue application.

It is desirable to provide a means of assuring that the same tissue sample volume is repeatably sampled, thus minimizing sampling errors due to mechanical tissue distortion, specular reflectance, and probe placement. It would also be desirable to provide a way to minimize temperature fluctuations and stabilize stratum corneum moisture content at the tissue measurement site, thus eliminating further sources of sampling error. It would also be highly advantageous to provide a coupling medium to provide a constant interface between an optical probe and the skin at a tissue measurement site that is non-toxic and non-irritating and that doesn't introduce error into spectroscopic measurements. Additionally, it is advantageous to provide a means of monitoring surface pressure at the tissue measurement site, therefore assuring that the sample probe placement minimizes tissue distortion of a sample site. Automated coupling fluid delivery, controlled methodology of sample probe placement, and control of thermal variation eases the use of a noninvasive glucose concentration analyzer and benefits a mechanical delivery system in terms of accuracy and repeatability.

### SUMMARY OF THE INVENTION

A coupling medium such as an optical coupling fluid, placed on the surface of tissue at a tissue measurement site, is used to enhance performance of an optical analyzer coupled to the tissue measurement site. Means of assuring that the same tissue sample volume is repeatably sampled are presented, thus minimizing sampling errors due to mechanical tissue distortion, specular reflectance, and/or probe placement. An automated coupling fluid delivery system improves accuracy and precision of the delivery of the fluid while facilitating the ready use of a noninvasive glucose concentration analyzer.

### DESCRIPTION OF THE FIGURES

FIG. 1 presents an analyzer comprising a base module, a sample module, and communication means according to the invention;

FIGS. 2 and 2B provide a perspective view (FIG. 2A) and an end view (FIG. 2B) of a fluid delivery system according to the invention;

FIG. 3 provides a block diagram of fluid delivery to a sample site according to the invention;

FIG. 4 provides a block diagram of fluid delivery to a sample site according to the invention;

FIG. 5 illustrates a potential energy assisted coupling fluid delivery system according to the invention; and

FIGS. 6a-6c illustrate a temperature controlled fluid delivery system according to the invention.

### DETAILED DESCRIPTION OF THE INVENTION

Sampling is controlled to enhance analyte concentration estimation derived from noninvasive sampling. In a first embodiment of the invention, sampling is controlled using automated delivery of a coupling fluid to a region between a tip of a sample probe and a tissue measurement site. In a second embodiment of the invention, sampling is controlled by controlling temperature variations at a region about the tip of a sample probe and a sample site. Details of particular embodiments of the invention are described, infra.

Analyzer

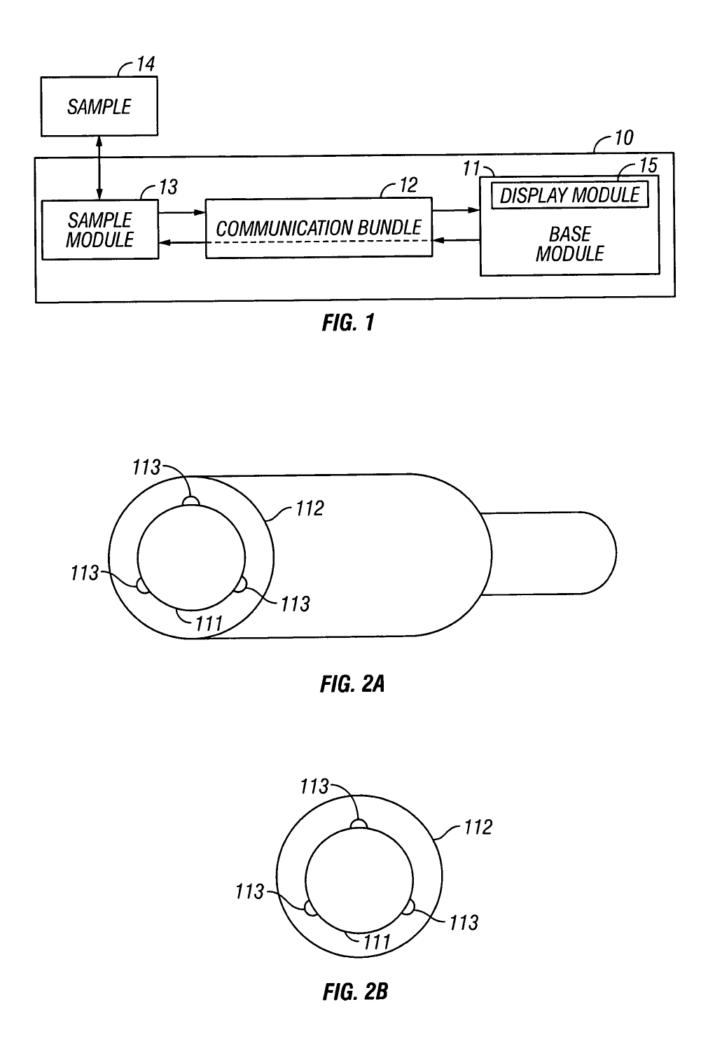

In many embodiments of the invention, an analyzer or a glucose tracking system is used. Referring now to FIG. 1, a block diagram of an analyzer 10 including a base module 11 and sample module 13 connected via communication means 12, such as a communication bundle is presented.

The analyzer preferably has a display module 15 integrated into the analyzer 10 or base module 11. The system uses a glucose concentration analyzer that comprises at least a source, a sample interface, at least one detector, and an associated algorithm.

Conventionally, all of the components of a noninvasive glucose analyzer are included in a single unit. Herein, the combined base module 11, communication bundle 12, sample module 13, and processing center are referred to as a spectrometer and/or analyzer 10. Preferably, the analyzer 10 is physically separated into elements including a base module in a first housing 11, a communication bundle 12, and a sample module in a second housing 13. Advantages of separate units include heat, size, and weight management. For example, a separated base module allows for support of the bulk of the analyzer on a stable surface, such as a tabletop or floor. This allows a smaller sample module to interface with a sample, such as human skin tissue. Separation allows a more flexible and/or lighter sample module for use in sampling by an individual. Additionally, separate housing requirements are achievable for the base module and sample module in terms of power, weight, and thermal management. In addition, a split analyzer results in less of a physical impact, in terms of mass and/or tissue displacement, on the sample site by the sample module. The sample module, base module, communication bundle, display module, and processing center are further described, infra.

Sample Module

A sample module 13, also referred to as a sampling module, interfaces with a tissue sample and at the same or different times with one or more reference materials. The sample module includes a sensor head assembly that provides an interface between the glucose concentration tracking system and the patient. The tip of the sample probe of the sample module is brought into contact or proximate contact with the tissue sample. Optionally, the tip of the sample probe is interfaced to a guide, such as an arm-mounted guide, to conduct data collection and removed when the process is complete. An optional guide accessory includes an occlusion plug that is used to fill the guide cavity when the sensor head is not inserted in the guide, and/or to provide photo-stimulation for circulation enhancement. In one example, the following components are included in the sample module sensor head assembly: a light source, a fiber optic, and coupling fluid. Preferably, the sample module is in a separate housing from the base module. Alternatively, the sample module is integrated into a single unit with the base module, such as in a handheld or desktop analyzer.

Communication Bundle

A communication bundle 12 is preferably a multi-purpose bundle. The multi-purpose bundle is a flexible sheath that includes at least one of:
electrical wires to supply operating power to the lamp in the light source;
thermistor wires;
one or more fiber-optics, which direct diffusely reflected near-infrared light to the spectrograph;
a tube, used to transport coupling fluid and/or optical coupling fluid from the base unit, through the sensor head, and onto the measurement site;
a tension member to remove loads on the wiring and fiber-optic strand and/or to moderate sudden movements; and
photo sensor wires.

Further, in the case of a split analyzer the communication bundle allows separation of the mass of the base module from the sample module as described herein. In another embodiment, the communication bundle is in the form of wireless communication. In this embodiment, the communication bundle includes a transmitter, transceiver, and/or a receiver that are mounted into the base module and/or sample module.

Base Module

A portion of the diffusely reflected light from the site is collected and transferred via at least one fiber-optic, free space optics, or an optical pathway to the base module. For example, a base module contains a spectrograph. The spectrograph separates the spectral components of the diffusely reflected light, which are then directed to a photo-diode array (PDA).

The PDA converts the sampled light into a corresponding analog electrical signal, which is then conditioned by the analog front-end circuitry. The analog electrical signals are converted into their digital equivalents by the analog circuitry. The digital data is then sent to the digital circuitry where it is checked for validity, processed, and stored in non-volatile memory. Optionally, the processed results are recalled when the session is complete and after additional processing the individual glucose concentrations are available for display or transfer to a personal computer. The base module also, preferably, includes a central processing unit or equivalent for storage of data and/or routines, such as one or more calibration models or net analyte signals. Preferably the base module includes a display module. In an optional embodiment, a base module includes one or more detectors used in combination with a wavelength selection device, such as a set of filters and/or a movable grating.

Display Module

A noninvasive glucose analyzer preferably contains a display module 15 that provides information to the end user or professional. Preferably, the display module 15 is integrated into the base module 11. Optionally, the display module is integrated into the sample module 13 or analyzer 10. The display screen communicates current and/or historical analyte concentrations to a user and/or medical professional in a format that facilitates information uptake from underlying data. A particular example of a display module is a 3.5″ VGA 320×240 pixel screen. The display screen is optionally a color screen, a touch screen, a backlit screen, or is a light emitting diode backlit screen.

Coupling Medium

The interface between an optical probe and a skin surface at the tissue measurement site is potentially a significant source of sampling error. There are a number of sampling issues including:
skin surface irregularity;
air gaps; and
refractive index mismatch.

These issues are distinct, but have some interrelationships. Skin surface irregularity results in an increase in the surface reflection of incident light. Basically, incident light normal to the surface penetrates into the skin sample based upon the difference in refractive index according to Snell's Law. For the refractive index of skin, approximately 1.38, and the refractive index of air, approximately 1.0, approximately 4% of the light is reflected and 96% of the light penetrates into the skin. The surface irregularities of skin mean that the incident light is not normal to the surface. This results in more reflected light, and less penetrating light. Air gaps near the skin surface complicate this issue. Some light penetrating into an outermost layer of skin hits an air pocket. Some light is reflected off of each surface of the air pocket. Many air pockets or poor hydration leads to a significant reduction in the percentage of incident photons that penetrate through the outermost skin layers, such as the stratum corneum, to the inner skin layer.

Coupling fluid use between a sample site and an interfacing sample probe surface is useful for a number of reasons. First, coupling fluid aids in reduction of surface reflection due to optical aberrations in surface coupling and stretching of the surface tissue due to sample probe contact. Second, the use of coupling fluid allows sample probe placement relative to the tissue site with minimal applied pressure to the sample site. Third, coupling fluid use aids in stabilizing hydration of surface tissue.

The refractive index mismatch and Snell's Law explain part of the effects described for the skin surface irregularities and air gaps. However, a coupling fluid need not be a refractive index matching fluid, also known as an optical coupling fluid, to increase light throughput. For example, in the case of a high refractive index material, such as a lens, coming into contact with skin via a coupling fluid, the coupling fluid need not have a refractive index between that of skin and the optic to be beneficial. For example, the percentage of incident photons passing through a silicon lens into skin is increased even with use of a coupling fluid that does not have a refractive index between that of silicon and skin. For example, a fluorocarbon, such as FC-40 manufactured by 3M Corporation, (St. Paul, Minn.) has an index of refraction of 1.290 that is not between that of skin, 1.38, and silicon, approximately 2. However, the FC40 still increases incident photon penetration by displacement of air. Specifically, for coupling silicon and skin FC-40 is not an index-matching medium, optical coupling fluid, or refractive-index matching coupling fluid.

However, it still aids in light coupling by displacing the lower refractive index air. Alternatively, a coupling fluid, such as a chlorofluorocarbon with a higher index of refraction, is called an index-matching medium. A chlorofluorocarbon with an index of refraction between that of the coupling medium and the skin increases the number of penetrating photons due to both index of refraction matching and displacement of the air that results in a smoother surface.

Coupling the relatively smooth surface of an optical probe with the irregular skin surface leads to air gaps between the two surfaces. The air gaps create an interface between the two surfaces that adversely affects the measurement during sampling of tissue due to refractive index considerations as described, infra. A coupling medium is used to fill these air gaps. Preferably, for an application, such as noninvasive glucose concentration estimation, the coupling fluid:
is spectrally inactive;
is non-irritating
is nontoxic;
has low viscosity for good surface coverage properties;
has poor solvent properties with respect to leaching fatty acids and oils from the skin upon repeated application; and
is thermally compatible with the measurement system.

It is possible to achieve these desirable characteristics by selecting the active components of the coupling fluid from the classes of compounds called fluorocarbons, perfluorocarbons, or those molecules containing only carbon and fluorine atoms. Nominally limiting chain length to less than twenty carbons provides for a molecule having the requisite viscosity characteristics. Generally, smaller chain lengths are less viscous and thus flow over the sample surface more readily. Longer chains are more viscous and tend to coat the sample surface with a thicker layer and run off of the sample site over a longer period of time. The molecular species contained in the perfluorocarbon coupling fluid optionally contain branched, straight chain, or a mixture of both structures. A mixture of small perfluorocarbon molecules contained in the coupling fluid as polydisperse perfluorocarbons provides the required characteristics while keeping manufacturing costs low. Additives are optionally added to the fluid.

In one embodiment, the coupling fluid is a perfluoro compound, such as those known as FC-40 and FC-70, manufactured by 3M Corporation (St. Paul, Minn.). This class of compounds is inactive in the near-infrared region, rendering them particularly well suited for sampling procedures employing near-infrared spectra. Additionally, they have the advantage of being non-toxic and non-irritating, thus they can come into direct contact with living tissue, even for extended periods of time, without posing a significant health risk to living subjects. Furthermore, perfluoro compounds of this type are hydrophobic and are poor solvents. Therefore they are unlikely to absorb water or other contaminants that adversely affect the resulting optical sample. It is preferable that the sampling fluid be formulated without the addition of other substances, such as alcohols or detergents, which may introduce artifacts into the optical sample. Finally, the exceptional stability of perfluoro compounds eliminates the environmental hazard and toxicity commonly associated with chlorofluorocarbons.

Other fluid compositions containing perfluorocarbons and chlorofluorocarbons are also suitable as coupling fluids: for example a blend of 90% polymeric chlorotrifluoroethylene and 10% other fluorocarbons have the desired optical characteristics. Chlorotrifluoroethene is optionally used. While these compositions have the desired optical characteristics, their toxicity profiles and their solvent characteristics render them less desirable than the previously described perfluoro compounds.

Additionally, other fluid media are suitable for coupling of an optical probe to a tissue measurement site, for example, skin toner solutions or alpha hydroxy-acid solutions.

Operation

During use, a quantity of sampling fluid is placed at the interface of the tissue measurement site and the fiber optic probe so that the tissue measurement site and the fiber optic probe are coupled leaving no or minimal air spaces between the two surfaces. Several methods of delivery sequence are described, infra.

A first method of coupling the interface of a tissue measurement site and a tip of a sample probe is to place a small amount of coupling fluid on the skin surface prior to placing the fiber optic probe in close proximity or in contact with the sample site.

A second method of coupling the interface of a tissue measurement site and a tip of a sample probe is to place coupling fluid on the tip of the sample probe and bring the sample probe into contact with a surface proximate the skin sample site.

A third method of coupling a tissue measurement site to an analyzer is to spray the tissue sample site with the coupling fluid and/or to spray the tip of the sample module and/or bundle prior to bring the sample into contact or close proximity with the analyzer.

A fourth method of coupling a measurement site to a tip of a sample module is to deliver the coupling fluid while the tip of the sample module is in motion. For example, coupling fluid is delivered through small tubes that terminate at the tip of the sample module near the area of photon delivery and/or near the area of photon collection. For example, a fluorocarbon is dropped onto the tissue sample site through tubes terminating next to a central collection fiber.

A fifth method of coupling a tissue measurement site and a tip of a sample probe is to provide channels or ridges in the tip of a sample probe that allow excess coupling fluid to be pushed out of the way or to drain off through gravity. A primary intent of this embodiment is to prevent applying undue pressure to the sample site when the tip of the sample probe is brought into close proximity and/or contact with the sample site. Pooling of excess coupling fluid is prevented by these channels. For example, a hydraulic effect created by the sample module pressing on coupling fluid on its way to the sample site is relieved by having channels through which excess coupling fluid flows when pressurized.

A sixth method of coupling the interface between the tissue measurement site and the tip of a sample probe is to first bring the tip of the sample probe into contact with the sample site, remove the sample probe from the sample site, deliver the coupling fluid, and then again bring the sample probe into close proximity with the sample site. This method eases locating the skin when a movable sample probe is used as described in U.S. provisional patent application No. 60/566,568, filed Apr. 28, 2004, which is incorporated herein in its entirety by this reference thereto. In addition, the elapsed period of time between coupling fluid delivery and optical sampling (measurement) is minimized thus reducing the risk of evaporation of the coupling fluid prior to sampling.

A seventh method is to pull a partial vacuum on or about a tissue sample site. For example, the tip of an optical probe is pulled away from the sample site after making contact. In a second example, the tip of tubing filled with a coupling fluid is in contact with a sample site and fluid is withdrawn from the tubing or is backed off from the tip of the tubing. This movement of the coupling fluid creates a partial vacuum. Creating a partial vacuum creates a small convex tissue meniscus. Fluid, such as interstitial fluid, flows into the meniscus. This results in increased concentration of the analytical target of interest in the sampled optical tissue. Alternatively, applying a small negative pressure reduces a negative meniscus making the sample more readily sampled with a flat optical surface.

An eighth method of applying coupling fluid to a tissue site is to warm the coupling fluid to a target temperature prior to application. Examples of target temperatures include about 88, 90, 92, 94, 96, and 98 degrees Fahrenheit. Optionally, the tip of the sample probe and/or surface of the sample site are adjusted to or toward this first target temperature or to their own target temperature. Preferably, the two target temperatures are the same in order to reduce sampling variations resulting from temperature variation. A variation is to independently control or not control the sample site, coupling optic, and coupling fluid temperature.

A ninth method of applying coupling fluid includes a step of removing coupling fluid from the sample site. Methods of removal include: waiting for a period of time to allow evaporation, allowing gravity induced run off of the fluid, and/or wiping off with a material, such as an absorbent cloth or wipe.

A tenth method of providing a coupling fluid between a tissue site and an optical probe is to apply coupling fluid multiple times. For example, one to ten microliters of coupling fluid is applied two or more times.

Optionally, coupling fluid is used to clean a sample site. For example, coupling fluid is applied to the sample site and removed as above in order to remove sample debris.

An eleventh method of providing coupling fluid between a tip or an end of a sample probe and a tissue site or sample site is to determine contact of a z-axis movable sample probe tip from a response signal, such as a pressure sensor, a response from a broadband source, or from a response to a photons emitted from a light emitting diode. For example, a light emitting diode is optionally used outside of the range detected by detectors coupled to a broadband source element in a sample module. For instance, the light emitting diode wavelength is centered at a spectral feature, such as due to water, fat, or protein, or within an optical window such as in the ‘H’, ‘J’, or ‘K’ band regions of the electromagnetic spectrum. An additional detector element is optically associated with the light emitting diode. For instance, a broadband source is used in conjunction with a grating from about 1100 to 1800 nm. A light emitting diode and its associated detector are used outside of the detected broadband source region to detect, through intensity change, contact of a sample probe, analyzer, or sample probe tip with a tissue sample. Particular water absorbance features that are optionally used occur at about 1900, 2000, or 2500 nm.

Combinations and permutations of the coupling fluid delivery methods described herein are also usable without diverting from the scope of the invention.

Furthermore, certain non-fluid media having the requisite optical characteristic of being near-infrared neutral are also suitable as a coupling medium, for example, a GORE-TEX membrane interposed between the probe and the surface of the measurement site, particularly when used in conjunction with one of the fluid media previously described.

Localized Delivery

Preferably, coupling fluid covers the entire sample site prior to sampling. Volume requirements for the various modes of delivery for a sample are small, such as less than about fifty microliters. Preferably five to thirty microliters of coupling fluid are applied to the sample site. For a sample site of about two to six millimeters in diameter, eight plus or minus one to two microliters is typically sufficient. Precision and/or accuracy of volume of delivery is important in order to avoid excess waste, sufficient coverage, and/or undue pooling. The target volume of delivery is dependent upon the sample probe geometry and size.

Coupling fluid is delivered at the sample site and/or near the sample site. As described herein, a number of methods of delivery exist including via spray, dribble, mist, gravity feed, capillary action, via peristaltic pump, magnet motor, or via a piston. Various modes of delivery apply the coupling fluid at or about the sample site. Referring now to FIGS. 2A and 2B, a perspective and end view of a particular embodiment of a fluid delivery system are presented, respectively. A central optic, such as a core and cladding of a fiber optic 111, are coated with a material 112. One or more lumens 113 are localized about the central optic 111 in the coating material 112. Coupling fluid is delivered to the sample site through the lumen 113. This system allows localized delivery of the coupling fluid to the sample site. Optionally, the central optic is a bundle of fiber optics or a single optic. The lumens 113 are optionally of any geometric shape, such as a circle, oval, triangle, square, or other polygonal shape. The lumens are either in contact with the central optic 111, are embedded in the coating material 112, or are located in close proximity to the coating material 112. Preferably, the lumens are extruded or co-extruded for ease of manufacture. The number of lumens in this example is optionally one or more. For example, two, four, or six lumens are used to deliver the coupling fluid to the sample site. The use of a larger number of lumens helps to insure coverage of the sample site by the coupling fluid.

Automated Delivery

An automated coupling fluid delivery system is used to deliver coupling fluid to a sample site with minimal human interaction. An automated coupling fluid delivery system provides many benefits including:
accurate fluid delivery volume;
precise fluid delivery volume;
accurate fluid delivery location;
precise fluid delivery location;
software controlled delivery;
delivery with minimal user input; and/or
ease of use.

Delivery of coupling fluid to a sample site is preferably performed by a lay user in a convenient manner. Automated control of one or more of the delivery steps is therefore preferential as the task is simplified for the user and controls to the delivery are established by the apparatus.

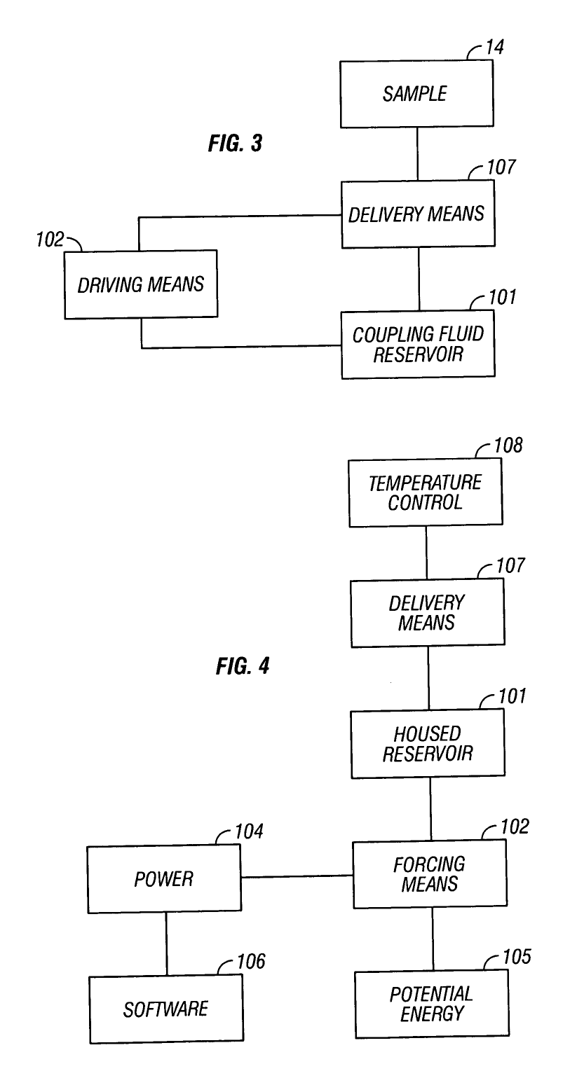

Referring now to FIG. 3, an example of a coupling fluid flow diagram is presented. A reservoir of coupling fluid 101 is moved to a sample site 14 via delivery means 107, such as tubing. Driving means 102 are used force the coupling fluid to the sample site 14. Examples of these elements are provided, infra.

Reservoir

A reservoir or container of coupling fluid is maintained so that a supply of coupling fluid is available for use with sampling. Maintaining a reservoir with the analyzer or having a reservoir integrated into the analyzer reduces the number of items that are independently handled by a user. This reduces the complexity of a noninvasive measurement and results in overall better performance in terms of accuracy and precision. Examples of reservoirs or containers include containers of various sizes, a syringe, a cartridge, a single use packet, a blister pack, a multiuse container, or a large auxiliary container. The reservoir is optionally a disposable or reusable. For instance, a small refillable reservoir is maintained within a sample module or within an analyzer. This allows, for example, the analyzer to be portable. In another instance, an external reservoir is coupled to the analyzer in either a permanent or removable fashion. Larger reservoirs are useful due to less frequent refilling requirements. Smaller reservoirs, such as a reservoir of less than one or two milliliters are still useful for multiple measurements as a preferred coupling fluid delivery volume is less than fifty microliters per use.

Delivery Means

Coupling fluid is moved from the reservoir to a sample site through delivery means, such as tubing, flexible tubing, or channels. The delivery means 107 optionally include a gate or a variable resistance flow section, especially when the housed reservoir is in close proximity to the sampling site. The coupling fluid is optionally routed through or integrated into a sample probe module. Optional routing through the sample module allows for delivery within close proximity to the sample site, such as within one inch. Delivery in an accurate area about a sample results in adequate coverage of the sample site while requiring less coupling fluid volume. For example, delivery near the sample site center allows about 5, 8, 10, 20, 30, or 40 microliters of coupling fluid to adequately cover the sample site. In addition, routing through the sample module allows movement of the sample module by a user to also control routing of the integrated delivery means without an additional action. In addition, the dual movement maintains tight control of the coupling fluid delivery to the sample site in terms or precision and accuracy of position of delivery. Precision and accuracy is further enhanced by the use of a guide coupling the sample module to the sample site. In an additional embodiment, the delivery channels or tubes run by thermal control means, such as a heat element, described infra. In still yet another embodiment, the delivery means 107 are thermally insulated.

Driving Means

Means are used to deliver coupling fluid to a sample site 14. Driving means 102 are available in a number of forms, such as via a motor, a solenoid, a gear, a piston, a peristaltic pump, gravity feed, capillary action, or a magnetic drive. Examples of magnetic drives are presented in U.S. patent application Ser. No. 10/752,369 which is incorporated herein in its entirety by this reference thereto. Power supplying the driving means include potential energy, electrical sources, manual force, gravity, and magnetic fields. Driving means optionally push or pull the fluid. Further, driving means are optionally connected to the reservoir 101 or to the delivery means 107.

Several examples of automated coupling fluid delivery systems are provided, infra.

### EXAMPLE I

Referring now to FIG. 3, a block diagram of an automated coupling fluid delivery system is provided. A coupling fluid is held in a reservoir 101. This reservoir contains the fluid in a package that allows for ready transport, protection from contaminants, and on-time delivery. An example reservoir is a syringe. Fluid is forced from the reservoir by driving means 102, such as a plunger. In this example a linear drive motor is used to move the plunger 102 into the syringe 101 and force coupling fluid through tubing 107 to the sample site 14.

Referring now to FIG. 4, optional power supplies 104 are used for powering the driving means 102 and include gravity and/or manual, alternating current, or direct current power. Often, the driving forces required tax a power budget. An optional potential energy assist 105 is provided to minimize auxiliary power requirements. Examples of a potential energy source 105 include a coiled spring or compressed gas, infra.

Optional software 106 is used to control coupling fluid delivery. The software is preferably tied into a larger data acquisition system of an analyzer, such as a central processing unit of a noninvasive glucose concentration analyzer. The software is used in either an open-loop or closed loop format. For example, the software controls delivery of a predetermined volume of coupling fluid to the sample site: The fluid delivery is preferably controlled by software to deliver at a set time within a sampling sequence, such as just prior to sampling skin tissue 14. Optionally, delivery volumes and/or times are controlled through software in a closed-loop system that has sensor feedback. Sensors include a contact sensor, a pressure sensor, and/or an optical signal such as a near-infrared spectrum.

### EXAMPLE II

In a second example of the invention, coupling fluid is delivered through tubing to a sample site. After delivery the coupling fluid is backed off from the end of the tubing exit, such as by capillary action or by reversing a pushing force into a pulling force. For example, a motor pushing the fluid is reversed and the fluid is pulled back a distance into the tubing. A sensor is optionally placed across the tubing to determine the position of the meniscus of the coupling fluid in the tubing. For example, a light source, such as a light emitting diode, shines through the tubing and is sensed by a detector. As first air and then coupling fluid is moved past the sensor in the tubing, a change in light intensity is indicative of the meniscus and hence the position of the coupling fluid in the tubing. The dead volume of tubing past the detector is readily calculated. The driving means 102, such as a stepper motor, are then used to deliver the dead volume of coupling fluid plus the desired volume of coupling fluid to be delivered to the sample site 14. In this manner, the desired delivery volume of coupling fluid is delivered to the sample site 14. Optionally, the motor is computer controlled. Optionally, there is a feedback between the detector response to the motor that provides a closed loop system controlling the volume of coupling fluid. Optionally, analyzer control software controls when coupling fluid is to be delivered to the sample site, such as after tissue has been sensed by the analyzer, after a hardware or software indication by the user, or at appropriate times in any of the methods in the Operation section, supra.

### EXAMPLE III

In a third example of the invention, a series of optical readings are collected by the analyzer. As the sample probe is brought into proximity to the sample site 14, the near-infrared reading changes. Features of the signal are indicative of the distance between the tip of the sample probe and the sample site. For example, the collected intensity at wavelengths of high absorbance decrease toward zero as the tip of the sample probe approaches a tissue sample. An example of a high absorbance feature is water at or about 1450 nm, 1900 nm, and/or 2600 nm. Correlation between intensity readings at one or more wavelengths and distance between the tip of the sample probe are used to provide feedback to the user or preferably to a z-axis moveable sample probe. The feedback allows the controller to move the sample probe relative to the tissue sample site. This allows, for example, controlling the probe to make contact with the sample, for the sample probe to be backed off from the sample, for a coupling fluid to be delivered to the sample site, and for the probe to be moved into close proximity to the sample probe, as described, supra. Examples of z-axis motor control of a sample module are described in U.S. provisional patent application No. 60/566,568, filed Apr. 28, 2004 which is incorporated herein in its entirety by this reference thereto. Optionally, the proximity between a tip of a sample module and a tissue site is determined with a pressure sensor placed on or near the tip of the sample probe of the analyzer. For example, contact is determined with the sensor or a proximate distance is determined by the feedback signal of the sensor.

Herein, an x, y, and z coordinate system relative to given a body part is used. The x-axis is along a body part, such as from an elbow to the wrist, from the shoulder to the elbow, or along the length of a digit of a hand. The y-axis moves across a body part. Together, the x,y plane tangentially touches the skin surface, such as at a sample site. The z-axis is normal to the x,y plane, such that an object moving toward the skin surface is moving along the z-axis. Thus a sample probe or portion of an analyzer brought toward a sample site is moving along roughly the z-axis.

### EXAMPLE IV

In a fourth example, the sample probe is moved in a manner that does not make contact with the tissue sample. Instead, the algorithm moves the tip of the sample probe into close proximity to the tissue sample before or after coupling fluid delivery and proceeds to sample the tissue with a small gap between the tissue sample and the tip of the optical probe. In this manner, pressure effects are alleviated and the coupling fluid reduces specular reflection to allow precise and accurate noninvasive glucose concentration estimations using near-infrared spectra. Optionally, in this embodiment the pathlength of the coupling fluid between the tip of the sample probe and the tissue sample is determined from an interference pattern. This interference pattern is then used to control the distance between the tip of the sample module and the tissue sample to a fixed pathlength.

### EXAMPLE V

In a fifth example of the invention, means are used to minimize formation of gaps in the delivery of coupling fluid to the sample site. For instance, air pockets or bubbles are preferably removed from a fluid delivery line. One optional mechanism for removing bubbles is an air trap. For instance, a larger piece of tubing or a small chamber where air can rise out of the flow line is used. Optionally, this line is bled off to remove air bubbles built up over time. In a second instance, the interior surface of the delivery means 107 is coated with a material that repels the coupling fluid. For example, a hydrophilic coating is placed on the interior of tubing. The hydrophilic coating repels fluorocarbons. Therefore, the fluorocarbon fluid sticks together instead of forming bubbles when the fluid is advanced or withdrawn through the tubing. Similarly, a hydrophobic surface is preferably used when moving a hydrophilic fluid, such as water.

### EXAMPLE VI

In a sixth example of the invention, an optional magneto-mechanical apparatus with a magnetic field modifier that, when inserted into or removed from the magnetic field between two magnet components of the apparatus, triggers a displacement of at least one of the magnet components, which is coupled to a drive or a switch. The drive 102 is used to move coupling fluid from a reservoir 101 through delivery means or channels 107 to the sample 14. The apparatus is based on coupled attracting or opposing magnets in conjunction with the insertion or removal of a magnetic field modifier. In one instance, two repelling magnets are drawn together with the insertion of a magnetic field modifier. The field modifier is optionally another magnet having an opposing pole. Removal of the field modifier returns the forces to their original states. This oscillating motion allows drive with a low energy and/or small power supply. The resulting motion of the opposing magnets is used to drive a mechanical system such as a linear, gear, ratchet, or reciprocating drive.

### EXAMPLE VII

A seventh example of an automated coupling fluid delivery system is software driving a solenoid with a direct current power supply assisted by a coiled spring. The solenoid drives a gear on a threaded plunger. The plunger forces fluid out of a syringe into tubing that is directed in proximity to a source filament where it is heated prior to delivery via tubing to a sample site.

### EXAMPLE VIII

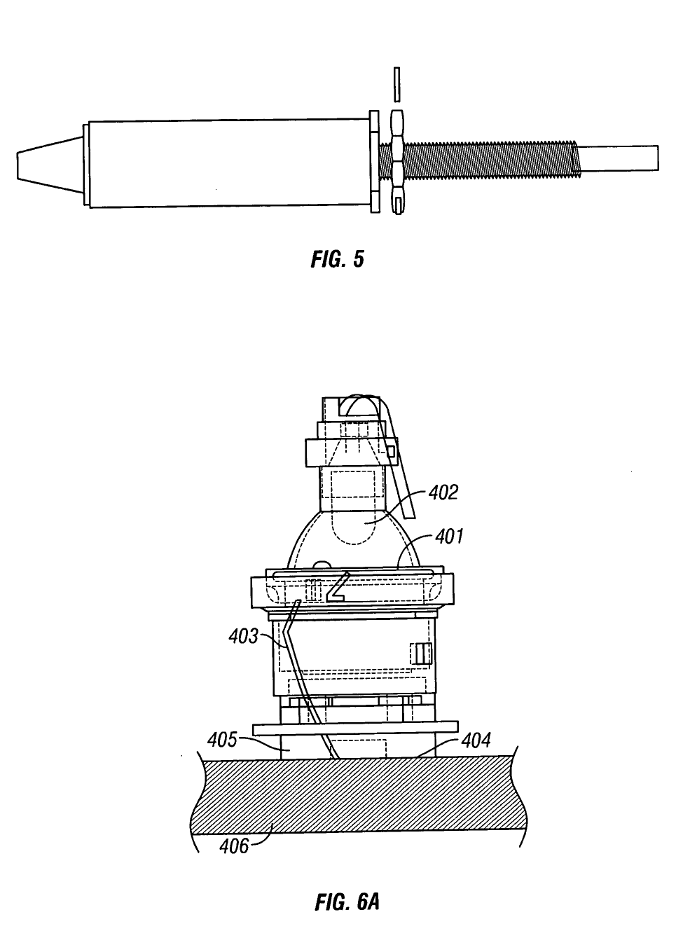

In an eighth example of the invention, potential energy is used as a power supply for driving coupling fluid to a sample site 14. Power 104 used to drive the coupling fluid is, optionally, assisted by a potential energy 105 power supply. The potential energy power supply pushes on the driving means 102. For example, a spring is affixed to a mechanical stop on one end and to the plunger on its opposite end. The coiled spring thus applies a potential energy force to the plunger thereby reducing the requirements of the driving means 102 and associated power supply 104. Another example of an alternative potential energy source is compressed gas. FIG. 5 provides an illustrative example of a potential energy assisted fluid delivery system. FIG. 5 illustrates a drive gear on a threaded plunger driven by external means. The driving of the plunger is assisted by a spring pressing on the plunger. The spring helps to drive the plunger and reduces back pressure from the fluid being delivered.

Thermal Control

In the case of a noninvasive measurement that uses an optic that contacts the sample site 14 skin during the measurement of the sample there is a potential for thermal variations due to conductive heat transfer between the skin and the contacting optic. Examples of optics in contact with the skin sample site include the tip of one or more fiber optics, a lens, or an optical window. Since conductive heat transfer is often very rapid and the effect of temperature on some near-infrared spectral features is large, the impact on the resulting spectrum is severe in some cases. For example, water that has large near-infrared absorbance bands is sensitive to temperature in both absorbance magnitude and wavelength of absorbance. Another example is near-infrared absorbances that relate to hydrogen bonding, which are known to be temperature sensitive. An exemplary noninvasive glucose concentration estimation case is when an optic at environmental ambient temperature is brought into direct contact with the skin surface, which is often at a much higher temperature than ambient temperature. In this case, a resulting sample spectrum has variation due to temperature variation due to heat transfer from the skin to the optic. In another case, heat is transferred from the optic to the skin, which also results in sample site temperature variation, which manifests in noninvasive spectra. Often the temperature variation manifested in the spectrum degrades subsequent analytical performance based upon use the spectrum.

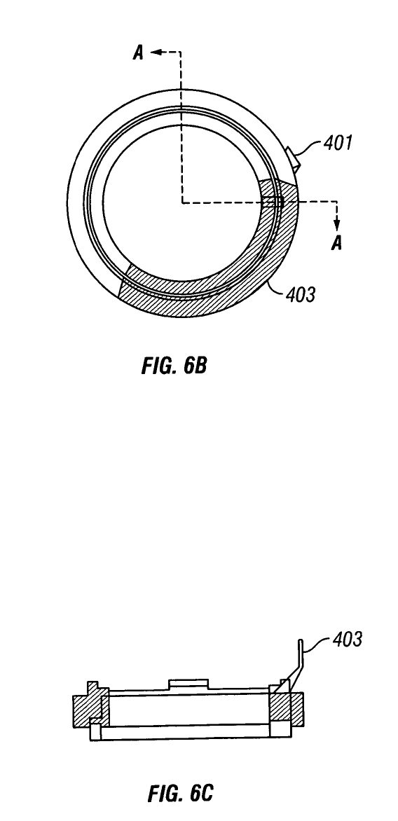

Temperature control of the skin contacting optic to a target temperature, such as the temperature of skin, minimizes thermal deviations in the measurement of the resulting spectrum. Optional temperature control is preferably performed on one or more of a sample probe tip, sample, reference material, and coupling fluid. For example, just the tip of a sample probe temperature is controlled to about skin surface temperature. In a second example, coupling fluid is preheated to a target temperature, such as about 85, 87, 89, 91, 93, 95, or 97 degrees Fahrenheit. In a third example, a two-stage system is used that uses one mechanism to control the skin temperature and another to control the optic to the targeted skin temperature. In a fourth example, a coupling fluid is thermally controlled and the warmed coupling fluid is applied to the sample site. This prevents a thermally cool coupling fluid site from locally cooling the sample site upon application of the fluid to the sample site. In a fifth example, an active heater 108 with feedback control is used to control the optic to a target temperature and/or to control the temperature of a coupling fluid to the target temperature. In a sixth example, a thermal stability fluid and/or a coupling fluid is used to control both the skin and the optic temperature. The temperature control point is ideally set closer to the skin temperature, as opposed to the ambient temperature, as the tissue sample typically has a much greater thermal mass compared with the contacting optic. A further example of a target temperate is ninety degrees Fahrenheit plus or minus two to three degrees Fahrenheit, which represents a natural physiological mean skin temperature in a targeted ambient measurement range of 63 to 82° F. An example of implementation is to first adjust the skin to a target temperature with a coupling fluid or first stage heater and second to control an interfacing optic to the target temperature. Subsequently, the optic is brought into contact with the sample. Optionally, the reference temperature is controlled to the temperature of the sample. This allows for a background that is representative of the thermal environment of the sample. An optional temperature control 108 device is used. A number of elements are optionally used for thermal control including auxiliary heating elements. Examples include a heat source, such as a filament, a heating strip, and a thermoelectric heater. Optionally, an internal heating element, such as an analyzer source, is used to provide heat to an optic, coupling fluid, and/or a target tissue site. For example, the high temperature source of the analyzer heats passing coupling fluid in a passive manner through heat transfer. The fluid in turn is used to cool the apparatus. For example, U.S. patent application Ser. No. 10/472,856, teaches a sample module that optionally contains a source. The source heats the module. The outer surface of the module is preferably kept cool so that it is readily handled. Coupling fluid directed near or around the source helps to cool the sampling module at the same time that the coupling fluid is brought to a thermal state that is compatible with sampling requirements. An example of a heating system is provided in FIGS. 6A-6C. Coupling fluid is delivered from the reservoir 101 to an input 401. The fluid is forced about a high temperature filament 402 to an exit 403. The fluid is delivered to an interface 404 between a sampling module 405 and the skin tissue 406.

While the invention is described in terms of noninvasive glucose concentration estimation, the methods and apparatus described herein also apply to estimation of additional tissue or body concentrations or properties, such as those associated with water, protein, fat, and urea. Further, while the invention is described in terms of near-infrared analysis from 1100 to 2500 nm, the methods and apparatus described apply to spectroscopic techniques ranging in the electromagnetic spectrum from the ultraviolet through the far infrared and to additional spectroscopic techniques, such as those based upon Raman or fluorescence spectroscopy.

Those skilled in the art will recognize that the present invention may be manifested in a variety of forms other than the specific embodiments described and contemplated herein. Departures in form and detail may be made without departing from the spirit and scope of the present invention. Accordingly, the invention should only be limited by the Claims included below.

1. An apparatus for delivery of coupling fluid to a sample site in connection with a noninvasive analyte concentration analyzer, comprising:
a reservoir coupled to said analyzer for containing said coupling fluid;
delivery means for periodically coupling said reservoir to said sample site; and
means for driving said coupling fluid from said reservoir to said sample site.

2. The apparatus of claim 1, wherein said reservoir comprises any of:
a replaceable cartridge;
a multiuse container;
a single use packet; and
a syringe.

3. The apparatus of claim 1, wherein said analyzer comprises:
a sample module in a first housing, said sample module having a tip;
a base module in a second housing separated from said first housing; and
a communication bundle having a first end connected to said sample module and a second end connected to said base module.

4. The apparatus of claim 3, wherein said reservoir resides in any of:
said analyzer;
said base module;
said sample module; and
a third housing separated from said analyzer.

5. The apparatus of claim 1, wherein said delivery means comprises any of:
tubing having an inner side;
flexible tubing;
a lumen;
routing; and
a channel.

6. The apparatus of claim 5, wherein said inner side of said tubing comprises a hydrophilic surface.

7. The apparatus of claim 3, wherein said delivery means routes said coupling fluid to within one inch of said tip of said sample module.

8. The apparatus of claim 1, wherein said analyzer further comprises:
means for temperature control.

9. The apparatus of claim 3, wherein said means for temperature control modify temperature of any of:
said sample site;
said tip of said sample analyzer; and
said coupling fluid.

10. The apparatus of claim 9, wherein said means for temperature control adjust temperature any of said sample site surface, said tip of said analyzer, and said coupling fluid toward a target temperature.

11. The apparatus of claim 10, wherein said target temperature comprises any of about 88, 90, 92, 94, 96, and 98 degrees Fahrenheit.

12. The apparatus of claim 1, said analyzer further comprising:
fluid detection means coupled to said delivery means.

13. The apparatus of claim 12, wherein said fluid detection means comprises:
a light source and a detector optically coupled to said light source via said delivery means for detection of intensity changes.

14. The apparatus of claim 1, said analyzer further comprising:
a processing unit integrated into said analyzer.

15. The apparatus of claim 1, wherein said analyzer further comprises:
optical means for alignment of said analyzer to said sample site.

16. The apparatus of claim 15, said optical means comprising:
a detector for outputting a signal, wherein said detector comprises any of:
a pressure sensor; and
a photon detector.

17. The apparatus of claim 15, said optical means comprising:
a z-axis movable sample probe.

18. The apparatus of claim 15, wherein said optical means comprises:
a closed-loop system.

19. The apparatus of claim 1, wherein said means for driving comprises any of:
gravity feed;
capillary action;
a peristaltic pump;
a motor;
a piston;
a drive;
a solenoid;
a gear;
potential energy; and
a magnetic drive.

20. The apparatus of claim 19, wherein said potential energy comprises any of:
a spring; and
compressed gas.

21. The apparatus of claim 1, wherein said means for driving comprises any of:
an automated delivery system; and
a closed-loop system.

22. The apparatus of claim 1, wherein said means for driving deliver less than twenty microliters of coupling fluid to said sample site with each use.

23. A method of sampling a tissue site, comprising the steps of:
providing a near-infrared noninvasive analyte concentration analyzer having a sample probe, said sample probe having an end;
sampling said tissue site with said analyzer, thereby generating signal;
estimating proximity of said sample probe end relative to said tissue site using said signal; and
dispensing coupling fluid about said tissue site based upon said proximity, wherein said coupling fluid is dispensed through said sample probe.

24. The method of claim 23, wherein said signal comprises any of:
an optical reading;
a near-infrared optical response;
a pressure reading; and
an interference fringe.

25. The method of claim 23, wherein said step of dispensing proceeds after said step of estimating proximity establishes proximate contact of said sample probe end with said tissue site.

26. The method of claim 23, further comprising moving said sample probe relative to said tissue site.

27. The method of claim 26, wherein said step of dispensing proceeds after said step of moving said sample probe retracts said tip of said sample probe from contact with said tissue site.

28. The method of claim 23, wherein said step of dispensing recurs after said step of moving said sample probe.

29. The method of claim 26, wherein said step of dispensing occurs during said step of moving said sample probe.

30. The method of claim 26, wherein said step of moving comprises at least z-axis movement of said sample probe tip.

31. The method of claim 23, further comprising a step of preheating an element.

32. The method of claim 31, wherein said element comprises any of:
a surface of said sample site, wherein said surface proximately contacts said end of said sample probe during said step of sampling;
said coupling fluid; and
said sample probe tip.

33. The method of claim 31, wherein said step of preheating comprises preheating:
said coupling fluid; and
said sample probe tip.

34. The method of claim 32, wherein said step of preheating comprises preheating to a target temperature, wherein said target temperature comprises any of about 88, 90, 92, 94, 96, and 98 degrees Fahrenheit.

35. The method of claim 23, wherein said step of sampling comprises collecting a noninvasive spectrum of said sample site; and further comprising a step of:
estimating analyte concentration from said noninvasive spectrum, wherein said analyte comprises any of:
glucose;
water;
fat; and
urea.

36. The method of claim 23, wherein said steps of estimating proximity and dispensing coupling fluid comprise any of:
an automated delivery system; and
a closed-loop system.

37. An apparatus for noninvasive estimation of an analyte property of a human with an analyzer, wherein a portion of said analyzer comprises a sample module having an end, said estimation performed via a sample site of said human, comprising:
a reservoir either connected to or integrated into said analyzer; and
means for automated delivery of coupling fluid between said reservoir and said sample site;
wherein at least a portion of said means for automated delivery is integrated with said analyzer.

38. The apparatus of claim 37, wherein said reservoir comprises either a replaceable cartridge or a multiuse container.

39. The apparatus of claim 37, wherein said means for automated delivery comprise either a manual control open-loop system or a closed-loop system.

40. The apparatus of claim 39, wherein said closed-loop system comprises any of:
signal input;
algorithm control;
z-axis movement control of said sample module; and
driving means.

41. The apparatus of claim 40, wherein said signal comprises any of:
an optical reading;
a near-infrared optical response;
a pressure reading;
temperature control; and
an interference fringe.

42. The apparatus of claim 40, wherein said signal comprises at least two of:
an optical reading;
a near-infrared optical response;
a pressure reading;
temperature control; and
an interference fringe.

43. The apparatus of claim 40, wherein said driving means comprises any of:
gravity feed;
capillary action;
a peristaltic pump;
a motor;
a piston;
a drive;
a solenoid;
a gear;
potential energy; and
a magnetic drive.

44. The apparatus of claim 42, wherein said temperature control comprises preheating any of:
a surface of said sample site, wherein said surface proximately contacts said end of said sample probe during use;
said coupling fluid; and
said end of said sample probe.

45. The apparatus of claim 44, wherein said preheating comprises heating to about any of about 88, 89, 90, 91, 92, 93, 94, 95, 96, 97, and 98 degrees Fahrenheit.

46. The apparatus of claim 44, wherein said property comprises concentration and said analyte comprises any of:
glucose;
water;
fat; and
urea.

47. The apparatus of claim 37, wherein said means for automated delivery routes within one inch of said end of said sample module and wherein sampling error is minimized, eliminated, reduced, or compensated.

48. The apparatus of claim 37, wherein said means for automated delivery deliver less than thirty microliters of coupling fluid to said sample site with each use.

49. A method for noninvasively sampling a tissue site having a surface, comprising the steps of:
providing a noninvasive analyte property analyzer having a sample probe, said sample probe having a tip;
setting a target temperature;
adjusting toward said target temperature at least two of:
said sample probe tip temperature;
said surface of said tissue site temperature; and
a coupling fluid temperature prior to application of said coupling fluid between said sample probe tip and said tissue site;
moving said sample probe tip into close proximity with said surface of said tissue site;
collecting noninvasive near-infrared signal of said tissue site with said analyzer; and
estimating said analyte property using said signal.

50. The method of claim 49, wherein said target temperature comprises any of about 88, 89, 90, 91, 92, 93, 94, 95, 96, 97, and 98 degrees Fahrenheit.

51. The method of claim 50 wherein said analyte comprises any of:
water;
protein;
fat;
urea; and
glucose.

52. An apparatus for noninvasively estimating a sample property with a near-infrared noninvasive analyte property analyzer having a sample probe, said sample probe having a tip through a tissue site having a surface, comprising:
means for adjusting toward a target temperature at least two of:
said sample probe tip temperature;
said surface of said tissue site temperature; and
a coupling fluid temperature prior to application of said coupling fluid between said sample probe tip and said tissue site;
means for moving said sample probe tip into close proximity with said surface of said tissue site, wherein said means for moving are integrated with said analyzer; and
means for noninvasive near-infrared signal collection representative of said tissue site with said analyzer.
